(19) United States
(12) Patent Application Publication
(10) Pub. No.: US 2007/0202492 A1
(43) Pub. Date: Aug. 30, 2007

(54) Viral Assay

(75) Inventors: Andrew John Easton, Warwickshire (GB); Anthony Colin Marriott, Kenilworth (GB)

Correspondence Address: BIRCH STEWART KOLASCH & BIRCH, PO BOX 747, FALLS CHURCH, VA, 22040-0747, US

(73) Assignee: University Of Warwick, Coventry (GB)

(21) Appl. No.: 10/599,904

(22) PCT Filed: Apr. 14, 2005
(86) PCT No.: PCT/GB05/01431

§ 371 Date: Oct. 12, 2006

(30) Foreign Application Priority Data
Apr. 15, 2004 (GB) 0408424.0

(51) Int. Cl. C12Q 1/70; C12Q 1/68

(52) U.S. Cl. 435/5; 435/6

(57) ABSTRACT

The application relates to an assay method for studying the effect of at least one compound on RN virus replication and transcription comprising the steps of providing a synthetic RN molecule encoding at least a portion of the genome of an RN virus of interest and a copy of a reporter gene; incubating a cell containing the RN molecule with the or each compound and detecting an amount of reporter gene product. Preferably the RNA virus is a paramyxovirus such as human respiratory syncytial virus or avian pneumovirus. The assay may be automated to allow the screening of large numbers of compounds for anti-viral activity. Kits for carrying out the assay method are also disclosed.

The application relates to an assay for studying the effect of at least one compound on RNA virus replication. The RNA virus may especially be a negative-strand RNA virus. The RNA virus may especially be a paramyxovirus including for example human respiratory syncytial virus (RSV) or avian pneumovirus (APV).

Viruses may exist in a number of forms. They may exist as single-stranded DNA, such as parvovirus, single-stranded circular DNA, such as M13, double-stranded DNA, such as herpes virus, double-stranded circular DNA, such as SV40, or as RNA viruses. RNA viruses may exist as double-stranded RNA, such as reovirus or single-stranded RNA. Single-stranded RNA viruses are known to exist in two forms. Positive-strand RNA viruses comprise an RNA genome which may be translated into protein directly. That is, they have RNA genomes that correspond to mRNA and can function as messages even in vitro. Many other RNA viruses have negative or minus(−) strand genomes, meaning they are complementary to the sense or mRNA strand. Since animal cells lack enzymes to copy RNA, and since the negative strands cannot be translated, negative strand RNA alone is not infectious. Viruses with negative strand genomes must encode an RNA-dependent RNA polymerase that can make positive sense RNA, including mRNA and a full length copy of the genome, from a negative strand RNA template. Furthermore, the enzyme must be packaged in the virion in association with the viral genome. After entry of the virus into the host cell, the genome-associated RNA-dependent RNA polymerase synthesises viral mRNA, allowing the replication cycle to begin. The viral RNA-dependent RNA polymerase recognises specific regulatory sequences which direct transcription and replication (D M Knipe and P M Howley, Fields Virology, 4th edition, Lippincott Williams & Wilkins, 2001). At the end of the cycle, newly synthesised molecules of RNA-dependent RNA polymerase are again packaged along with the genome, making their next cycle of infection possible. RNA virus genomes are packed by encapsidation of the genome RNA by virus-encoded proteins which recognise one or more virus-specific nucleic acid sequences in the genome RNA (Knipe & Howley, Fields Virology, 2001 Supra).

Positive sense single-stranded RNA viruses include retroviruses such as HIV; picomaviruses such as rhinoviruses and foot and mouth disease virus; flaviviruses such as yellow fever virus, West Nile virus, dengue virus and hepatitis C virus; alphaviruses such as sindbis virus, and equine encephalitis viruses; and coronaviruses such as the SARS virus, as well as numerous plant viruses.

Negative strand RNA viruses include the rhabdoviruses which cause rabies and vesicular stomatitis. Other examples are paramyxoviruses, which include Newcastle disease virus, measles virus, mumps virus, respiratory syncytial virus (RSV), avian pneumovirus (APV—also known as turkey rhinotracheitis virus) and Sendai virus; orthomyxoviruses, which cause influenza; and bunyaviruses, which cause among other diseases Rift Valley fever.

Human RSV is the leading viral etiologic agent of serious infant respiratory tract disease, causing bronchiolitis and pneumonia in young children. This leads to the hospitalisation of 10-12,000 children per year in the United Kingdom alone. It is also an infection of adults and can kill the weak or old. By the age of 10 years it is thought that everyone has been infected at some time by RSV.

APV is a major disease of turkeys and causes a large amount of economic damage to the turkey breeding industry.

Currently, there is no vaccine against RSV available. The anti-viral agent ribavirin is known to act upon RSV in cell culture. However, it is rarely used clinically since the compound may have major side effects and is not always effective. Therapeutic compounds in clinical use include Respigam® and Synagis® which contain neutralising antibodies.

Current assays for testing antiviral compounds against negative strand RNA viruses are labour-intensive. For RSV, methods used include plaque assay and measurement of cytopathic effect (Kawana, F. et al., Antimicrobial Agents and Chemotherapy, 31, 1225-1230, 1987; Watanabe, W. et al., Journal of Virological Methods, 69, 103-111, 1994).

Park, K. H. et al (PNAS USA, 88, 5537-5541, 1991) disclose the construction of RNA molecules containing a negative sense copy of a reporter gene. The negative sense RNA molecule may simply comprise a 3′ leader sequence containing a promoter for synthesising positive-sense RNA, attached to the negative sense copy of the reporter gene, and a 5′ trailer sequence. The reporter gene is flanked by viral regulatory sequences to direct transcription by the viral RNA polymerase. The negative sense minigenome RNA molecule is simply introduced into a cell containing Sendai virus and incubated. The Sendai virus within the cell encodes the viral RNA-dependent RNA polymerase necessary for converting the negative sense RNA molecule into positive sense RNA, so that the reporter gene is in a form in which it may be expressed. The virus minigenome was demonstrated to be packaged into infectious particles. This art has been applied to several other viruses and especially paramyxoviruses as described by Marriott, A. C. and Easton, A. J. (Advances in Virus Research, 53, 312-340, 1999).

Schnell M. J. et al (EMBO Journal, 13, 4195-4203, 1994) disclose the generation of an infectious rabies virus which contains an altered gene. The virus was derived from a plasmid which can be used to direct synthesis of a positive sense copy of the negative sense RNA genome. The plasmid was introduced into cells expressing the N, P and L proteins of rabies virus. Expression of the altered gene was detected following replication and transcription of the virus. This art has been applied to several other viruses and especially paramyxoviruses as described by Marriott, A. C. and Easton, A. J. (Advances in Virus Research, 53, 312-340, 1999) and by Conzelmann, K. K. (Annual Review of Genetics, 32, 123-162, 1998).

Olivo, P. D. et al (Virology, 251, 198-205, 1998) and U.S. Pat. No. 6,270,958 disclose an assay for the detection and quantitation of RSV. They used BHK cells which had been transformed with a Sindbis virus replicon expressing bacteriophage T7 RNA polymerase. These cells were then cotransfected with T7 expression plasmids that contain the cDNA of an RSV minigenome and the genes for RSV nucleocapsid proteins N, P and L. The minigenome contained a reporter gene such as chloramphenicol acetyl transferase (CAT) flanked by cis-acting RSV replication and transcription signals. Subsequent infection of these cells with RSV resulted in a high level of reporter gene expression which could be inhibited by ribavirin. The assay is complicated and is not readily amendable to automation for large-scale screening of compounds.

U.S. Pat. No. 6,376,171 discloses an assay showing that one specific protein, M2-1 of RSV can be a target for antiviral compounds. The transcript of the product is assayed and it does not use a heterologeous reporter gene.

Positive-strand RNA vectors and replicons based on, for example, Sindbis virus are also known (Agapov, E. V., et al. (1998), 95, 12959-12994). Such vectors and replicons have again been used as naked, non-infectious, non-packaged RNA. Alpha virus expression vectors have also been demonstrated by Frolov, I., et al. (PNAS (USA) (1996), 93, 11371-11377). This paper shows the amplification of alpha virus replicons, and also discusses packaging the virions, for example to allow targeting of engineered alpha viruses to specific cell types or to incorporate heterologous ligands or receptors into the virion envelope. This paper also reviews the production of such replicons as virus vectors for gene therapy. No mention is made of using the vectors to assay pro- or anti-viral compounds.

WO 03/63783 discloses the use of viral replicons containing reporter genes. While this technique has been described for the assay of antiviral agents, it does not allow virus entry, encapsidation and maturation to be assayed. This technique specifically avoids the use of infectious virus particles.

Lo, M. K., et al. (J. Virol. (2003), 77 (33), 12901-12906) discloses an assay for screening inibitors of West Nile virus. This involves transcribing replicon DNA into RNA in vitro. This RNA is transfected into BHK cells and the cells are grown in a selective medium. Cell lines containing the replicons are then screened for the presence of an antibiotic resistance gene and reporter gene. The cloned cells are then placed into wells and screened for reporter gene activity. This process is very labour intensive and does not use infectious viral particles. Only drugs that target replication are assayed, not those that target entry, encapsidation or maturation.

The inventors have realised that a synthetic copy of an RNA genome containing a reporter gene could be used to assay for anti-viral agents, including chemical compounds and antibodies, and small interfering RNAs (siRNA) in a screening process which is amenable to automation using RNA molecules which have been packaged into infectious particles. The assay relies on the detection of the reporter gene expression as a measure of virus RNA-dependent RNA polymerase activity.

Collins, et al., as long ago as 1991, (PNAS USA 88, 9663-9776, 1991) used an assay of the reporter gene to study the effects of various sequences within the genome of RSV on virus replication. However, the inventors have unexpectedly recognised that the assay may be used to detect new compounds having either a positive or a negative effect on viral replication. Despite the length of time the previous use has been carried out, the new use is not previously known. The inventors have realised that the assay could be readily automated to enable large numbers of different compounds to be tested. The assay also allows the activity of compounds on virus entry, uncoating, replication and encapsidation to be assayed at the same time. This has the potential to assay for a broader range of compounds than prior art assays.

Accordingly, a first aspect of the invention provides an assay method for studying the effect of at least one compound on RNA virus entry, RNA replication, transcription or encapsidation, the method comprising the steps of:
(a) providing an RNA molecule encoding (i) at least a portion of the genome of an RNA virus of interest, (ii) a copy of a reporter gene flanked by viral regulatory sequences to direct transcription by a viral RNA polymerase and (iii) one or more sequences of RNA encoding packaging signals, the RNA molecule being packaged within a virus-like particle;
(b) incubating a cell containing the RNA molecule with the or each compound, the cell being capable of causing the replication of the RNA molecule; and
(c) detecting the presence of any reporter gene product.

Preferably, the RNA virus is a negative-strand RNA virus. Alternatively, it may be a positive-strand RNA virus.

Preferably the RNA is produced by (i) introducing an RNA molecule encoding (1) at least a portion of the genome of an RNA virus, such as a positive-strand or a negative-strand RNA virus of interest, (2) a copy of a reporter gene flanked by viral regulatory sequences to direct RNA synthesis by the viral RNA-dependent RNA polymerase and (3) one or more sequences of RNA encoding packaging signals, into a cell infected with the cognate virus; or (ii) introducing a plasmid capable of directing the synthesis of the RNA molecule into a cell infected with the cognate virus and containing the components required to enable to plasmid to direct synthesis of the RNA; or (iii) introducing a plasmid capable of directing the synthesis of the RNA molecule containing the genes necessary for virus replication and packaging into a cell containing the components required to enable to plasmid to direct synthesis of the RNA and containing the components required for viral replication and transcription.

The RNA molecule in step (i) may be positive- or negative-sense RNA. The virus may be a positive or negative sense single stranded RNA virus.

The viral RNA polymerase is preferably encoded by the genome of the RNA virus.

The term “negative sense RNA molecule” means an RNA molecule which is complementary to the sense or mRNA strand. That is, the negative sense RNA molecule cannot be translated without being first converted into positive, sense, RNA.

The term “virus-like” means that the RNA molecule is packaged into an infectious particle, for example in a similar manner to wild-type RNA for wild-type virus. The RNA may be packaged with one or more coat proteins and may produce an infectious particle.

Preferably, the RNA molecule in step (a) is packaged within a virus-like particle and the virus-like particle is used to infect cells step (b).

The use of a packaging signal on the RNA molecule enables the molecule to be packaged within the infectious viral particles. The secreted, packaged molecule may then simply be collected and used to infect cells to test for anti- or pro-viral compounds.

Such packaging signals and regulatory signals are known (see e.g. Knipe and Howley, Supra).

The use of a packaged RNA enables the use of infectious viral particles to introduce the RNA into cells. This allows the system to be automated without the need to use complicated transfection systems.

The virus used need not be the same strain used to make the RNA molecule.

The RNA copy of the reporter gene is preferably operatively linked to a suitable virus promoter, so that RNA synthesis from the RNA molecule generates a positive sense mRNA in the cell, and the reporter gene may be expressed. The reporter gene may be any RNA sequence encoding a detectable gene product. Alternatively, the newly synthesised RNA itself may be detected. Such RNA may be detected by techniques known in the art such as reverse transcription PCR, or Northern blots. Alternatively, the reporter gene may encode a polypeptide, such as protein or peptide, product. The polypeptide may be detected immunologically or by means of its biological activity. The reporter genes used may be any known in the art. The reporter genes are preferably heterologous to the cell in which the RNA molecule is replicating.

Preferably, the reporter gene is that for luciferase. Luciferase reporter genes are known in the art. They are usually derived from firefly (Photinus pyralis) or sea pansy (Renilla reniformis). The luciferase enzyme catalyses a reaction using D-luciferin and ATP in the presence of oxygen and Mg²⁺ resulting in light emission. The luciferase reaction is quantified using a luminometer which measures light output. The assay may also include coenzyme A in the reaction which provides a longer, sustained light reaction with greater sensitivity. The assay is amenable to automation.

An alternative reporter gene may be that for chloramphenicol acetyltransferase (CAT) which is well known in the art. CAT catalyses the transfer of the acetyl group from acetyl-CoA to the substrate chloramphenicol. The enzyme reaction can be quantified by incubating cells or cell lysates with [¹⁴C] chloramphenicol and following product formation by physical separation with, for example, thin layer chromatography or organic extraction. Alternatively, the CAT protein can be quantified using an enzyme-linked immunosorbant assay. Such an assay is available from, for example, Promega Corporation, Southampton, United Kingdom.

A further reporter system which may be used is lacZ gene from E. coli. This encodes the β-galactosidase enzyme. This catalyses the hydrolysis of β-galactoside sugars such as lactose. The enzymatic activity in cell extracts can be assayed with various specialised substrates, for example X-gal, which allow enzyme activity measurement using a spectrophotometer, fluorimeter or a luminometer.

The reporter gene may also be that for green fluorescent protein (GFP), which is also known in the art. The expressed GFP may be detected in a fluorimeter.

The reporter gene may also be that for secreted alkaline phosphatase. This is a known reporter gene which has the advantage that supernatant may readily be assayed for the enzyme. This lends itself to automation.

The cell may be any suitable cell which is capable of being infected with the RNA virus of interest and/or which is capable of supporting replication of the RNA molecule. It may be animal, such as mammalian, avian or insect, or a plant cell, depending on the RNA virus.

Methods of introducing nucleic acids into cells are well-known in the art.

Preferably the RNA molecule in step (a) or (b) is incapable of independent replication. That is, it is incapable of being copied into the complementary sense RNA, without the assistance of components provided from within the cell. In this case, the cell will contain components necessary to allow synthesis of mRNA from the RNA molecule leading to expression of the reporter gene. Such cells are well-known in the art.

The RNA molecule may lack one or more genes encoded by the genome, such as the genome of a negative-strand RNA virus. Indeed, all of the genes encoded by the original negative-strand RNA virus may have been deleted. This may simply leave suitable non-translated sequences to enable the RNA molecule to be replicated and transcribed into mRNA. Such sequences will include, for example, the 3′ leader sequence encoding a promoter for synthesising a positive-sense RNA.

Preferably, the virus is a positive-strand RNA, such as retroviruses such as HIV; picornaviruses such as rhinoviruses and foot and mouth disease virus; flaviviruses such as yellow fever virus, West Nile virus, dengue virus and hepatitis C virus; alphaviruses such as sindbis virus, and equine encephalitis viruses; and coronaviruses such as the SARS virus, as well as numerous plant viruses.

Preferably the virus is a negative-strand RNA virus such as a paramyxovirus. Such viruses include RSV and APV. Other paramyxoviruses may also be used such as parainfluenza type 3, measles virus and mumps virus. Other viruses of the order Mononegavirales may also be used.

By comparing the results of assays carried out with or without the compound of interest, it is possible to identify compounds having pro-viral or anti-viral activity.

The invention also provides a pro-viral or an anti-viral compound identified by the use of an assay according to the invention. The compound may be an antibody or an siRNA.

The assay method has considerable advantages over traditional assay methods. Plaque assay requires considerable operator experience, and not every strain of every virus is able to induce visible plaques. Immunostaining of plaques is a lengthy procedure and requires virus-specific antibody. Methods based on cytopathic effect require the virus to induce sufficient cell death in the cell type used, and will not work for less cytopathic viruses. The invention requires only a simple reporter assay which can readily be automated, and does not require the virus to induce cytopathic effect or plaques in the cells.

Kits for carrying out the assay method of the invention are also provided.

The invention will now be described by way of example with reference to the accompanying Figures.

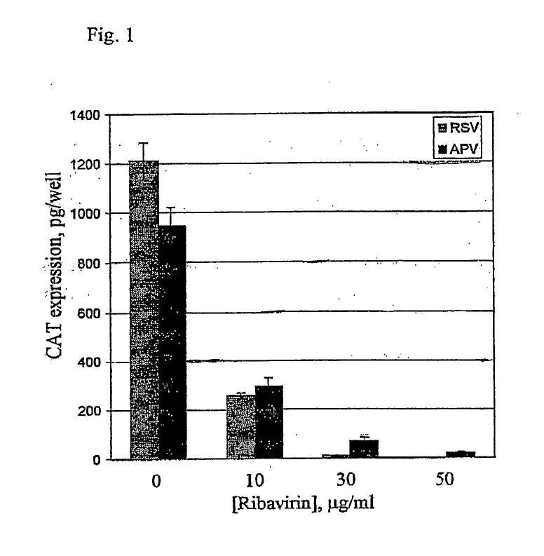

FIG. 1 shows the effect of ribavirin on CAT expression using an assay according to the invention. This is shown for both RSV and APV.

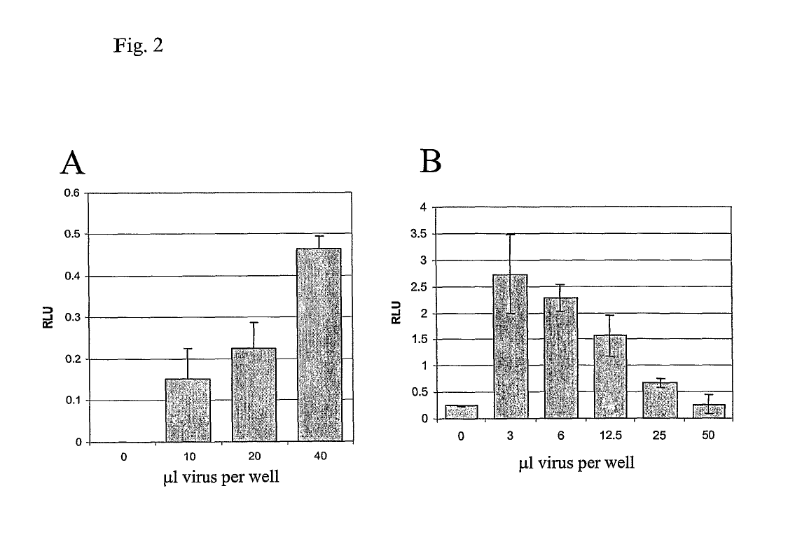

FIG. 2 shows the detection of RSV using SEAP as the reporter gene, and detection of PIV3 using luciferase as the reporter gene. Titration of virus stocks containing reporter genes in BS-C-1 cells (A) RSV-SEAP, (B) PIV3-Luciferase.

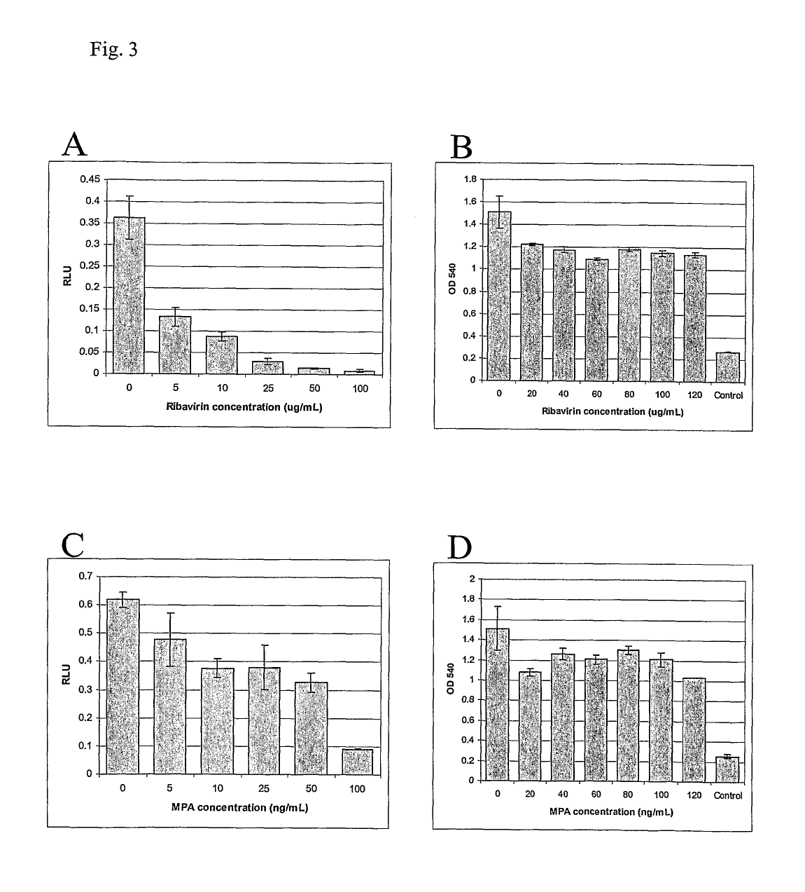

FIG. 3 shows the activity of ribavirin and mycophenolic acid on APV, using luciferase as the reporter gene according to the invention. (A) Ribavirin tested against APV-luciferase, (B) Ribavirin toxicity in BS-C-1 cells, (C) Mycophenolic acid tested against APV-luciferase, (D) Mycophenolic acid toxicity in BS-C-1 cells.

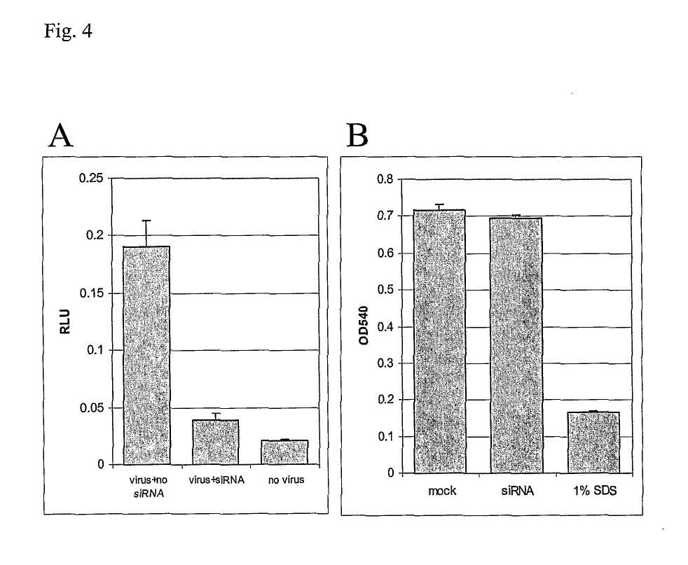

FIG. 4 shows the effect of siRNA specific for RSV on luciferase expression according to the invention. (A) RSV-luciferase reporter activity and (B) cell viability.

### METHODS

The methods exemplified herein may also be readily extended to other negative- and positive-strand RNA viruses.

Production of Viral Stocks Containing Minigenome.

The minigenome constructs have been described (Randhawa, et al., 1997). Each contains viral leader, gene start, CAT gene, gene end and viral trailer regions, in the direction 3′ to 5′. The APV-based minigenome is described in detail in Randhawa, et al. (1997). The RSV-based minigenome plasmid is described in Marriott, et al. (Journal of Virology, 75, 6265-6272, 2001). RNA transcripts were produced with T7 RNA polymerase and purified with Trizol reagent (Invitrogen).

To initiate a seed stock from the RSV minigenome, Vero cells in a 12-well culture plate were infected with RSV strain RSS-2 at 4-5 pfu/cell. After 1 hr at 37° C. the cells were transfected with 2 μg RNA using a lipid transfection reagent, such as Lipofectin (Invitrogen) or Fugene (Roche). After 3 days at 37° C., supernatant and cells were harvested. 1 ml supernatant was used to infect a 25 cm² flask of HEp-2 cells. After 48 hr at 37° C., cells and supernatant (5 ml) were harvested.

In the case of the APV minigenome, Vero cells in a 6-well culture plate were infected with APV strain CVL14/1 at 6 pfu/cell. Following transfection with 5 μg RNA, cells and supernatant were harvested after 3 days at 37° C. Vero cells in a 12-well culture plate were infected with 1 ml supernatant, and harvested after 3 days at 37° C. This second supernatant (1 ml) was used to infect a 25 cm² flask of Vero cells, and after a further 3 days at 37° C., cells and supernatant (5 ml) were harvested.

These stocks form the basis for the antiviral assay on microtitre plates.

Stocks of RSV and APV containing the luciferase gene or the secreted alkaline phosphatase (SEAP) gene were constructed as described above, except that the CAT coding region in the minigenome was replaced with the luciferase or SEAP coding region, respectively.

The PIV3 minigenome was constructed as described by Dimock & Collins (1993), except that the MK9 strain of human PIV3 was used as the source of the viral sequences, and luciferase replaced CAT as the reporter gene.

For some experiments, minigenome DNA was transfected into virus-infected cells rather than minigenome RNA. In this case, cells expressing T7 RNA polymerase were used to allow transcription of the minigenome RNA inside the cells.

Microtitre Plate Assay.

BS-C-1 cells were seeded into a 96-well microtitre plate. Wells were infected with 50 μl minigenome stock, or medium only. After a 1 hr adsorption period, supernatant was discarded and replaced with 200 μl medium containing 0, 10, 30 or 50 μg/ml ribavirin (Sigma). Each assay was performed in triplicate. The plate was incubated at 37° C. for 3 days. Supernatants were discarded, and the cells were washed with phosphate-buffered saline. Cells were lysed by adding 50 μl lysis buffer (1% Triton X-100, 10 mM MOPS, pH 6.5, 10 mM NaCl, 1 mM EGTA) to each well and incubating at room temperature for 30 min. The lysates were then transferred to the wells of a CAT ELISA plate (Roche), and the antigen-capture ELISA protocol was followed according to the manufacturer's instructions. The optical density at 405 nm was determined in a Labsystems Multiskan RC plate reader, and quantitation was performed using standards containing known amounts of CAT protein.

If the reporter gene was luciferase, an equal volume of SteadyGlo luciferase assay reagent (Promega) was added to the well. After 10 minutes, light emission was measured in a Luminoskan Ascent luminometer (Thermo Labsystems).

If the reporter gene was SEAP, the supernatant was removed from the well and incubated at 65° C. for 10 minutes. Aliquots of the supernatant were then mixed with CSPD reagent (Roche). After 10 minutes, light emission was measured in a Luminoskan Ascent luminometer.

Cell viability was measured using the MTT assay, exactly as described by Watanabe et al. (1994). For the negative control, cells were killed with 1% w/v SDS.

When using siRNA as the antiviral compound, the siRNA was transfected into the cells immediately following the virus adsorption step. The transfection reagent used was Lipofectamine 2000, according to the manufacturer's instructions (Invitrogen). The double-stranded siRNA was purchased from Invitrogen, and used as a 20 μM stock.

Results

Minigenome Stocks.

The quality of the stocks was determined by performing a CAT ELISA on the lysed cells from each passage (Table 1). Optimal amount of reporter-containing virus for use in the assay was determined by titration in a 96-well plate of BS-C-1 cells, as shown in FIG. 2.

Microtitre Plate Assay.

FIG. 1 shows the mean±standard deviation for each viral minigenome at each concentration of ribavirin. The drug has an obvious inhibitory effect on both viruses at the lowest concentration tested, namely 10 μg/ml. CAT expression is undetectable for RSV at 50 μg/ml ribavirin.

EC50 and EC90 values were calculated from several experiments for both viruses (Table 2).

These data agree well with the inhibitory activity of ribavirin on RSV as determined by reduction in plaque number on BS-C-1 cells, i.e. 100-fold reduction in titre due to 30 μg/ml drug. Published values using plaque assay or cytotoxicity assay in HeLa cells suggest an EC50 value of 3.1-6.2 μg/ml for ribavirin with RSV (Kawana, et al, 1987, Watanabe, et al., 1994) which agrees with the values obtained by the Inventors. Ribavirin has not previously been noted as an inhibitor of APV. The differences seen in FIG. 1 between RSV and APV responses do not necessarily imply that APV is less sensitive to ribavirin than is RSV, since the minigenorne stocks were not assayed for their viral titre, and it may be that the APV stock contains a higher titre of helper virus than does the RSV stock.

TABLE 1
Total CAT protein expression per dish or flask (pg).

| Virus + minigenome | Passage 0 | Passage 1 | Passage 2 |
|---|---|---|---|
| RSV | 3 405 | 7 931 | ND |
| (per 10⁶ cells) | 6 810 | 2 558 | ND |
| APV | 990 | 11 760 | 40 875 |
| (per 10⁶ cells) | 825 | 23 520 | 13 185 |

TABLE 2
50% and 90% effective concentrations
of ribavirus on pneumoviruses.

| | EC50 (μg/ml) | EC90 (μg/ml) |
|---|---|---|
| RSV | 4.2 | 17.1 |
| APV | 9 | 25.4 |

FIG. 2 demonstrates the use of reporter genes other than CAT. Enzyme activity is expressed as relative light units (RLU). In this case, the optimum amount of RSV containing SEAP as reporter was 40 μl per well (A), and the optimum amount of PIV3 containing luciferase (LUC) as reporter was 3 to 6 μl per well (B).

FIG. 3 shows the results of an antiviral assay using APV containing LUC reporter as the target virus, and ribavirin and mycophenolic acid (MPA) as the antiviral compounds. In agreement with the data shown in FIG. 1, APV is strongly inhibited by the higher concentrations of ribavirin (A). This demonstrates that the antiviral assay is not dependent on the nature of the reporter gene chosen. Panel (B) shows that ribavirin is not toxic to the cells at the concentrations used in the experiment. Panel (C) shows that MPA has antiviral activity against APV at 100 ng/ml, at which concentration the drug is not toxic to the cells (panel D). The control for cell toxicity in panels (B) and (D) is 1% SDS.

FIG. 4 shows the effect of an siRNA targeted against RSV in the antiviral assay. RSV containing LUC reporter was used. The siRNA sequence used was known to target the P gene of RSV (Bitko & Barik, 2001). Panel (A) shows that treatment with 100 nM siRNA resulted in reduction of the luciferase signal, almost to background level. Panel (B) shows an MTT assay for treatment of uninfected cells with 100 nM siRNA, and demonstrates that the siRNA was not cytotoxic. The control for cell toxicity was 1% SDS, which kills all the cells. Hence the reduction in luciferase activity seen in (A) must be a specific effect on RSV replication.

### REFERENCES

Randhawa, J. S., Marriott, A. C., Pringle, C. R. & Easton, A. J. (1997). Rescue of synthetic minireplicons establishes the absence of the NS1 and NS2 genes from avian pneumovirus. J. Virol. 71, 9849-9854.
Marriott, A. C., Smith, J. M. and Easton, A. J. (2001). Fidelity of leader and trailer sequence usage by respiratory syncytral virus and avian pneumoviruses replication complexes. Journal of Virology, 75, 6265-6272.
Conzelmann, K. K. (1998). Non-segmented negative-strand RNA viruses: genetics and manipulation of virus genomes. Annual Review of Genetics, 32, 123-162.
Kawana, F., Shigeta, S., Hosoya, M., Suzuki, H. and DeClerq, E. (1987). Inhibitory effects of antiviral compounds on respiratory syncytral virus replication in vitro. Antimicrobial Agents and Chemotherapy, 31, 1225-1230.
Watanabe, W., Konno, K., Ijichi, K., Inoue, H., Yokota, T. and Shigeta, S. (1994). MTT calorimetric assay system for the screening of anti-orthomyxo- and anti-paramyxoviral agents. Journal of Virological Methods, 48, 257-265.
Dimock, K. & Collins, P. L. (1993). Rescue of Synthetic Analogs of Genomic RNA and Replicative-Intermediate RNA of Human Parainfluenza Virus Type 3. Journal of Virology 67, 2772-8.
Bitko, V. & Barik, S. (2001). Phenotypic silencing of cytoplasmic genes using sequence-specific double-stranded short interfering RNA and its application in the reverse genetics of wild type negative-strand RNA viruses. BMC Microbiology 1, 34.

1. Assay method for studying the effect of at least one compound on RNA virus entry, RNA replication, transcription or encapsidation, the method comprising the steps of:
(a) providing an RNA molecule containing (i) at least a portion of the genome of an RNA virus of interest, (ii) a copy of a reporter gene flanked by viral regulatory sequences to direct RNA synthesis by a viral RNA polymerase and (iii) one or more sequences of RNA encoding packaging signals, the RNA molecule being packaged within a virus-like particle;
(b) incubating a cell containing the RNA molecule with the or each compound, the cell being capable of causing the replication of the RNA molecule; and
(c) detecting the presence of any reporter gene product.

2. Assay method according to claim 1, wherein the RNA virus is a negative strand RNA virus.

3. Assay method according to claim 1, wherein the RNA molecule is produced by (i) introducing an RNA molecule encoding (1) at least a portion of the genome of an RNA virus of interest, (2) a copy of a reporter gene flanked by viral regulatory sequences to direct RNA synthesis by the viral RNA polymerase and (3) one or more sequences of RNA encoding packaging signals, into a cell infected with the cognate virus; or (ii) introducing a plasmid capable of directing the synthesis of the RNA molecule into a cell infected with the cognate virus and containing the components required to enable the plasmid to direct synthesis of the RNA; or (iii) introducing a plasmid capable of directing the synthesis of the RNA molecule containing the genes necessary for virus replication and packaging into a cell containing the components required to enable the plasmid to direct synthesis of the RNA and containing the components required for viral replication and transcription.

4. Assay method according to claim 3, wherein the RNA molecule defined in step (i) is either negative-sense or positive-sense RNA.

5. Assay method according to claim 1, wherein the reporter gene is a heterologous reporter gene.

6. Assay method according to claim 1, wherein the RNA molecule is incapable of independent replication and the cell in step b contains components necessary for replication and packaging of the RNA molecule.

7. Assay method according to claim 1, wherein the RNA molecule may or may not lack one or more genes encoded by the native genome of the negative-strand RNA virus.

8. Assay method according to claim 1, wherein the negative-strand RNA virus is a paramyxovirus.

9. Assay method according to claim 8, wherein the negative-strand RNA virus is human respiratory syncytial virus (RSV), or avian pneumovirus (APV).

10. Assay method according to any preceding claim, wherein the reporter gene is chloramphenicol acetyltransferase (CAT), luciferase, green fluorescent protein (GFP), (β-galactosidase, or secreted alkaline phosphatase.

11. An antiviral or a proviral compound identified by use of an assay method according to claim 1.

12. A kit for use in an assay method according to claim 1 comprising an RNA molecule packaged into an infectious virus particle and encoding (i) at least a portion of the genome of an RNA virus, (ii) a copy of a reporter gene and (iii) one or more sequences of RNA encoding packaging signals.

13. A kit according to claim 12, wherein the RNA virus is a negative-strand RNA virus.

14. A kit according to claim 13, wherein the RNA virus is RSV or APV.

15. A kit according to claim 12, additionally comprising instructions for carrying out the assay method according to.
